(19) United States
(12) Patent Application Publication
(10) Pub. No.: US 2023/0098502 A1
(43) Pub. Date: Mar. 30, 2023

(54) Use Of Ionic Polymers For Hydrolysis Of Proteins And Protein-Containing Feedstock

(71) Applicant: EMBION TECHNOLOGIES SA, Etoy (CH)

(72) Inventors: Sviatlana SIANKEVICH, Fechy (CH); Georgios SAVOGLIDIS, Fechy (CH); Paul DYSON, Ecublens (CH); Johannes BANSCH, Ecublens (CH)

(21) Appl. No.: 17/796,947

(22) PCT Filed: Feb. 4, 2021
(86) PCT No.: PCT/EP2021/052597

§ 371 Date: Aug. 2, 2022

(30) Foreign Application Priority Data
Feb. 4, 2020 (EP) 20155416.9

(51) Int. Cl. C07K 1/12; C08F 226/06; A23J 3/32; A23J 3/08; A23J 3/14; A23J 3/20; A23L 33/18; A23L 33/175; A23L 33/19; A23L 33/185; A23L 33/195

(52) U.S. Cl. CPC C07K 1/12; C08F 226/06; A23J 3/32; A23J 3/08; A23J 3/14; A23J 3/20; A23L 33/18; A23L 33/175; A23L 33/19; A23L 33/185; A23L 33/195; A23V 2002/00

(57) ABSTRACT

The invention relates to use of ionic polymers (IP) consisting of anions and a polymeric backbone containing cations, in hydrolyzing proteins and protein-containing feedstock to produce protein hydrolysates.

### FIELD OF THE INVENTION

The invention relates to use of ionic polymers (IP) consisting of anions and a polymeric backbone containing cations, in hydrolyzing proteins and protein-containing feedstock to produce protein hydrolysate.

### BACKGROUND OF THE INVENTION

Proteins make up all of the body's organs and are required for proper function of organ systems. All proteins are made up of amino acids, but differences in amino acid composition and sequence differentiate how proteins function. The body uses amino acids to construct specific proteins for specific functions in the maintenance of organ health. However, the body has no de novo route for synthesis of many necessary amino acids, therefore these essential amino acids must be obtained from dietary protein. The body must consistently digest, absorb and metabolize adequate dietary proteins to supply organs with the specific proteins needed to function.

Dietary protein can be more easily digested and absorbed by consuming smaller peptide subunits of proteins or even a mixture of amino acids. Peptides and amino acids can be obtained by hydrolyzing proteins that often come from plant, animal, fungal or microorganism sources. Peptides and amino acids are usually obtained by acidic, alkaline or enzymatic hydrolysis of proteins. Alkaline hydrolysis process results in the complete destruction of most amino acids (almost 100% loss). Acid hydrolysis process offers the advantage of low cost. However, this process results in the complete destruction of tryptophan, a partial loss of methionine, and the conversion of glutamine into glutamate and of asparagine into aspartate. The main disadvantages of enzymatic hydrolysis of proteins is that pH and temperature ranges that optimize enzyme activity tend to maximize protein folding. High stearic hindrance results in poorly hydrolysed proteins, which adversely affects solubility, absorption, potency, sensory properties and interaction stability. Furthermore, enzymatic hydrolysis of protein includes the relatively high cost and the potential presence of enzyme inhibitors in the raw protein materials.

Industrial processes for hydrolysing proteins to produce protein hydrolysates comprising oligopeptides, peptides and/or amino acids rely mainly on enzyme mixtures which are far from optimal so that expensive purification steps are needed to produce peptide mixtures having sub-optimal size distributions.

Therefore, there is still a need for simple and safe method for hydrolysing proteins from various sources to produce protein hydrolysates of high digestibility, high absorption properties and high benefits.

### SUMMARY OF THE INVENTION

An aspect of the present invention provides a method for hydrolysing proteins and/or protein-containing feedstock into protein hydrolysate, the method comprising the steps of:

a) providing protein and/or protein-containing feedstock;

b) optionally determining protein content in the protein-containing feedstock;

c) optionally pre-treating the protein-containing feedstock;

d) optionally isolating proteins from the protein-containing feedstock to form a protein concentrate;

e) contacting the protein, the protein concentrate and/or the protein-containing feedstock with a catalyst to form a reaction mixture, wherein the catalyst is an ionic polymer or a combination of ionic polymers, the ionic polymer network, a solid-supported ionic polymers and/or a polymer membrane incorporating ionic polymers;

f) degrading the protein, the protein concentrate and/or the protein-containing feedstock in the reaction mixture to produce a liquid phase and a solid phase, wherein the liquid phase includes a protein hydrolysate, and the solid phase includes residual materials;

g) isolating at least a portion of the liquid phase from the solid phase; and

h) recovering the protein hydrolysate from the isolated liquid phase.

wherein the ionic polymer (IP) consists of a monomer of formula I

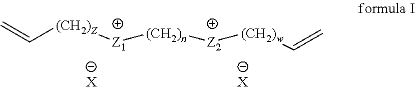

or consists of a first monomer of formula I

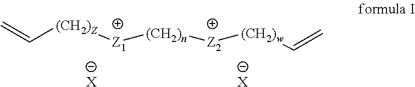

and at least one second monomer selected from the group consisting of

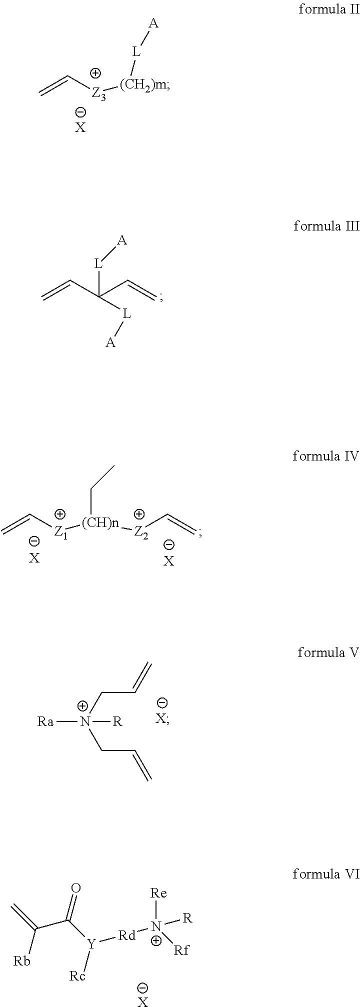

wherein
n and m are independently selected from 1, 2, 3, 4, 5, 6;
z and w are independently selected from 0, 1, 2, 3;
Z₁, Z₂ and Z₃ are cations each independently selected from the group comprising:

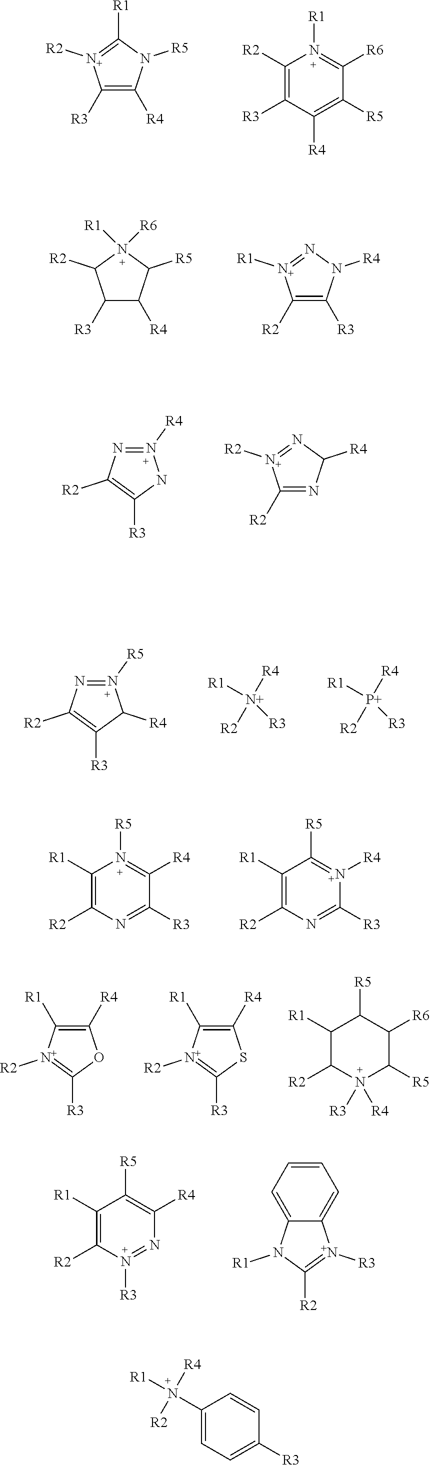

R1, R2, R3, R4, R5, R6 and R7 are each independently selected from the group comprising a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)];
A is an optional acidic group and each occurrence of A, if present, is independently selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], —CH₂—COOH, provided that when z and w are 0, A is present in formula IV;
X⁻ is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate;
Ra is C₁-C₂₄ alkyl;
Rb and Rc are each independently selected from the group comprising H and CH₃ or absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is selected from the group comprising C₁-C₂₄ alkyl and C₅-C₁₀ aryl or is absent.
wherein the ionic polymer network comprises cross-linked the one or more ionic polymers (IP);
wherein the solid support has at least one surface comprising the one or more ionic polymers (IP) or the ionic polymer network;
wherein the polymer membrane incorporates the one or more ionic polymers (IP) or the ionic polymer network.

Another aspect of the present invention provides the protein hydrolysate obtained by the method for hydrolysing proteins and/or protein-containing feedstock of the present invention.

Another aspect of the present invention provides a protein hydrolysate comprising peptides and free amino acids, wherein the protein hydrolysate is water soluble, having improved dissolving properties and degree of hydrolysis above 2.5% and wherein peptides are oligopeptides and polypeptides, optionally linked to carbohydrates, having a molecular weight of less than 10′000 Da.

Another aspect of the present invention provides a use of the protein hydrolysate of the present invention in the manufacturing of food products, animal feed products and cosmetic products.

Another aspect of the present invention provides a food product comprising an edible material and the protein hydrolysate of the present invention.

Another aspect of the present invention provides an ionic polymer (IP) consisting of a first monomer of formula I

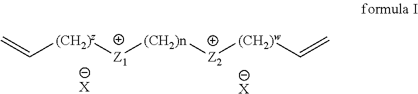

and at least one second monomer selected from the group consisting of

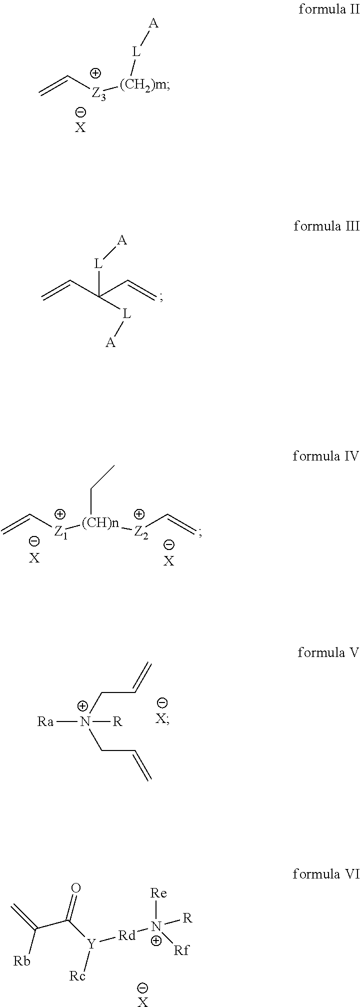

provided that at least the second monomer of formula V or at least the second monomer of formula VI is present in the ionic polymer (IP),

wherein
n and m are independently selected from 1, 2, 3, 4, 5, 6;
z and w are independently selected from 0, 1, 2, 3;
Z₁, Z₂ and Z₃ are cations each independently selected from the group comprising:

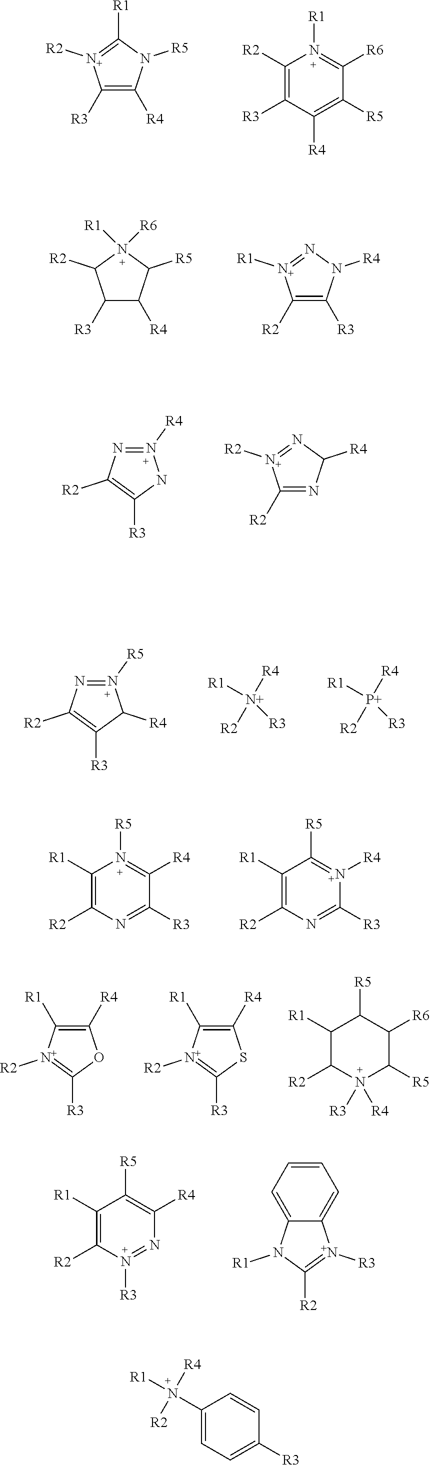

R1, R2, R3, R4, R5, R6 and R7 are each independently selected from the group comprising a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)];
A is an optional acidic group and each occurrence of A, if present, is independently selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], —CH₂—COOH, provided that when z and w are 0, A is present in formula IV;
X⁻ is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate;
Ra is C₁-C₂₄ alkyl;
Rb and Rc are each independently selected from the group comprising H and CH₃ or absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is selected from the group comprising C₁-C₂₄ alkyl and C₅-C₁₀ aryl or is absent.

### BRIEF DESCRIPTION OF THE FIGURES

FIG. 1 shows MS(+) chromatogram of the hydrolysed gluten mixture (top) and generated peptides profile (bottom).

FIG. 2 shows MS(+) chromatogram of the hydrolysed albumin mixture.

FIG. 3 shows MS(+) chromatogram of the hydrolysed spent barley mixture.

FIG. 4 shows growth of a Lactobacillus X strain in the presence of Glucose, FOS, XOS and the composition Prembion comprising protein hydrolysate of the invention. Colony counts as a function of growth time for a Lactobacillus X strain that has been reported to be responsible, in part, for human and animal gut health. All additives were added on a 1% (by total sugars basis) level.

FIG. 5 shows growth of a Bifidobacterium X strain in the presence of Glucose, FOS, XOS and the composition Prembion comprising protein hydrolysate of the invention. Colony counts as a function of growth time for a Bifidobacterium X strain that has been reported to be responsible, in part, for human and animal gut health. All additives were added on a 1% (by total sugars basis) level.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

An “allyl” group is a substituent with the structural formula H₂C═CH—CH₂R, where R is the rest of the molecule.

The term “monomer” refers to a molecule that can undergo polymerization or copolymerization thereby contributing constitutional units to the essential structure of a macromolecule (a polymer).

“Cross-linking”, as used herein, refers to the attachment of two or more monomers, oligomers or longer polymer chains by bridges of a cross-linker, such as an element, molecular group, a compound, or another oligomer or polymer. Cross-linking can result in a polymeric network (which can be two-dimensional or three-dimensional) where the polymer subunits are interconnected with multiple cross-linking agents and without free ends. Cross-linking may take place upon exposure to a stimulus, such as heat or light. As a result, some cross-linking processes occur at increased temperature, and some may also occur at room temperature or at lower temperature. As cross-linking density is increased, the properties of a material can be changed from thermoplastic to thermosetting.

The terms “peptide” or “oligopeptide”, as used herein, are defined as a chain of at least two amino acids that are linked through peptide bonds. The terms “peptide” and “oligopeptide” can be used interchangeably as the context requires.

A protein consists of one or more chain comprising of more than 30 amino acid residues (polypeptides) linked together by peptide bonds.

As used herein a “protein hydrolysate” (or “hydrolysate” or “hydrolysed protein”) is the product that is formed by hydrolysis of the protein peptide bonds between amino acids and it refers to a mixture of amino acids and peptides of different chain length. An enriched hydrolysate being a fraction of the protein hydrolysate, for example enriched in selected peptides or wherein a subset of peptides or polypeptides have been removed from the hydrolysate. So an enriched hydrolysate is preferably a mixture of peptides or a peptide mixture.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed).

As used herein, the term “functional food” refers to a food product to which a biologically active supplement has been added, such as a protein hydrolysate of the present invention.

As used herein, the term “nutritional supplement” refers to a food product formulated as a dietary or nutritional supplement to be used as part of a diet.

An aspect of the present invention provides a method for hydrolysing proteins and/or protein-containing feedstock into protein hydrolysate.

In an embodiment, the present invention provides a method for hydrolysing proteins and/or protein-containing feedstock into a protein hydrolysate, the method comprising the steps of:

a) providing protein and/or protein-containing feedstock;

b) optionally determining protein content in the protein-containing feedstock;

c) optionally pre-treating the protein-containing feedstock;

d) optionally isolating proteins from the protein-containing feedstock to form a protein concentrate;

e) contacting the protein, the protein concentrate and/or the protein-containing feedstock with a catalyst to form a reaction mixture, wherein the catalyst is an ionic polymer or a combination of ionic polymers, the ionic polymer network, a solid-supported ionic polymers and/or a polymer membrane incorporating ionic polymers;

f) degrading the protein, the protein concentrate and/or the protein-containing feedstock in the reaction mixture to produce a liquid phase and a solid phase, wherein the liquid phase includes the protein hydrolysate, and the solid phase includes residual materials;

g) isolating at least a portion of the liquid phase from the solid phase; and

h) recovering the protein hydrolysate from the isolated liquid phase.

In one embodiment, the step e) contacting the protein, the protein concentrate and/or the protein-containing feedstock with a catalyst to form a reaction mixture consists in adding water or an appropriate organic solvent and an effective amount of the catalyst to the protein, the protein concentrate and/or the protein-containing feedstock to form a reaction mixture, wherein the catalyst is an ionic polymer of the invention or a combination of ionic polymers of the invention, the ionic polymer network of the invention, a membrane incorporating ionic polymers of the invention and/or a solid-supported ionic polymers of the invention; and degrading step f) consists in heating the reaction mixture of step e) during appropriate time and subsequently cooling to room temperature (typically 20-25° C.). In some embodiments, the heating is between 50° to 170° C. or 100° C. to 160° C. or 110° C. to 150° C. or 140° C. to 160° C. or 100° C. to 170° C. In some other embodiments heating is at maximum 50° C. or at maximum 100° C., or at maximum 170° C. In other embodiments, the appropriate time is typically 0.5 to 3 hours. The reaction temperature and time depend on the origin of the protein or protein-containing feedstock.

In some embodiments of the method for hydrolysing proteins and protein-containing feedstock into protein hydrolysate of the present invention, the method further comprises applying a pressure of N₂ or CO₂ during the degrading step f). The pressure may range from 20 bar to 300 bar, preferably from 20 to 150 bar.

The term “protein”, as used herein, refers to any animal, plant and microorganism protein.

The term “protein-containing feedstock”, as used herein, refers to living or dead biological material that can be used in the method for producing the protein hydrolysate of the present invention. In some embodiments, the protein-containing feedstock is selected from the group comprising plant-based protein, single-cell protein, in vitro meat, yeast extract, spent yeast or yeast slurry, spent barley, insects, soybeans, pea, rapeseed, whey, casein, wheat, canola, corn, Jatropha, palm, peanut, sunflower, coconut, mustard, cotton seed, Palm kernel, olive, safflower, sesame, linseed, algae, crustaceans, fish meal, meat and bone meal, molasses, sprouted grains and legumes, collagen and other main commercial categories of protein isolates.

Optionally, prior to any use, protein content is determined in the protein-containing feedstock according the Bradford protein assay, Kjeldahl's method or Lowry method.

The optional pre-treatment of the protein-containing feedstock, used in the methods described herein, uses one or more methods selected from the group consisting of washing, solvent-extraction, solvent-swelling, comminution, milling, steam pre-treatment, explosive steam pre-treatment, dilute acid pre-treatment, hot water pre-treatment, alkaline pre-treatment, lime pre-treatment, wet oxidation, wet explosion, ammonia fibre explosion, organosolvent pre-treatment, biological pre-treatment, ammonia percolation, ultrasound, electroporation, microwave, supercritical CO₂, supercritical H₂O, ozone, and gamma irradiation. The optional pre-treatment of the protein-containing feedstock includes for example the milling of the protein-containing feedstock.

The optional isolating proteins from the protein-containing feedstock to form a protein concentrate, used in the method described herein, uses one or more selective precipitation methods selected from the group consisting of salting out; isoionic precipitation; organic co-solvent precipitation; two carbon (C₂) organic co-solvent precipitation of proteins; C₄ and C₅ organic co-solvent precipitation, phase partitioning and extraction of proteins; protein exclusion and crowding agents (neutral polymers) and osmolytes; synthetic and semisynthetic polyelectrolyte precipitation; metallic and polyphenolic heteropolyanion precipitation; hydrophobic ion pairing (HIP) entanglement ligands; matrix-stacking ligand co-precipitation; di- and trivalent metal cation precipitation.

Some ionic polymers used in the method of the invention for hydrolysing proteins and protein-containing feedstock into protein hydrolysate consists of anions and a polymeric backbone containing cations as disclosed in WO 2019/058270 A1 incorporated by reference in its entirety. Specifically, the ionic polymer (IP) used in the method of the invention for hydrolysing proteins and protein-containing feedstock into protein hydrolysate consists of a monomer of formula I

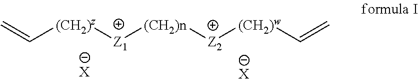

or consists of a first monomer of formula I

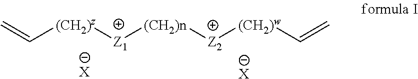

and at least one second monomer selected from the group consisting of

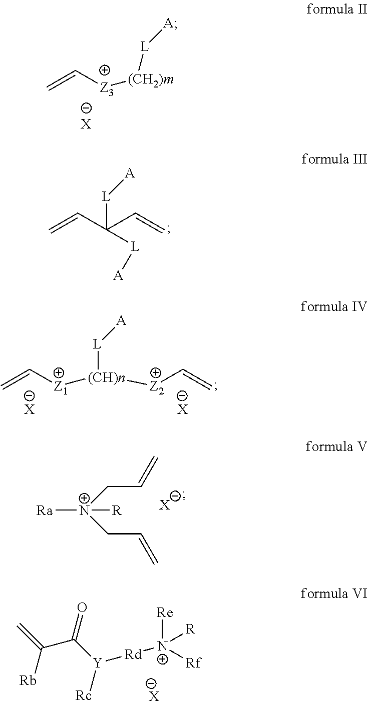

wherein
n and m are independently selected from 1, 2, 3, 4, 5, 6; preferably n and m are independently selected from 1, 2, 3; most preferably n is 2 and m is 1 or 2.
z and w are independently selected from 0, 1, 2, 3; preferably z and w are independently selected from 0 and 1; most preferably z and w are 0 or 1.
Z₁, Z₂ and Z₃ are cations each independently selected from the group comprising:

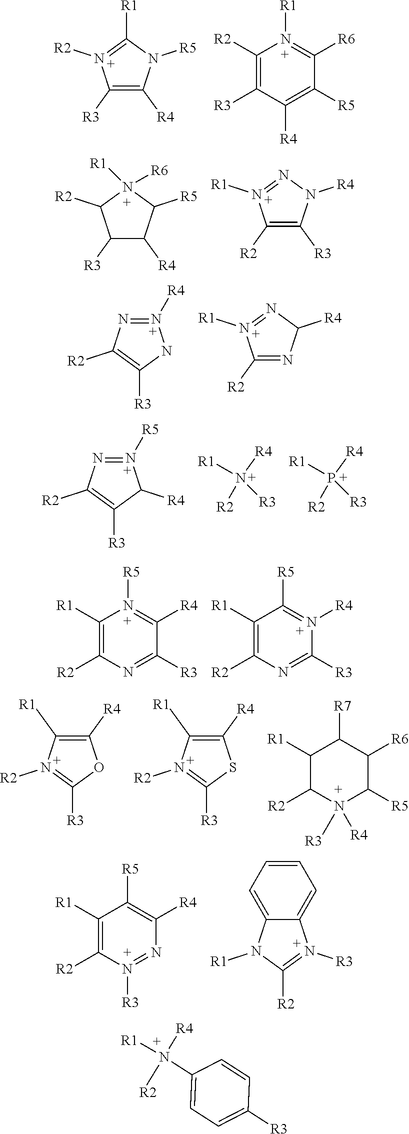

preferably Z₁, Z₂ and Z₃ are cations each independently selected from the group comprising:

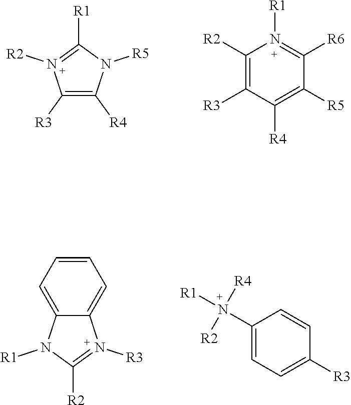

most preferably Z₁, Z₂ and Z₃ are cations each independently selected from the group comprising:

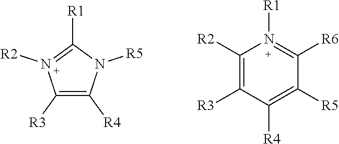

R1, R2, R3, R4, R5, R6 and R7 are each independently selected from the group comprising a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond; preferably R1, R2, R3, R4, R5, R6 and R7 are each independently selected from the group comprising a bond, H, C₁-C₆ alkyl, provided that two of R1, R2, R3, R4, R5, R6 and R7 are each a bond; most preferably R1, R2, R3, R4, R5, R6 and R7 are each independently selected from the group comprising a bond and H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted C₁-C₂₀ alkylene, C₁-C₂₀ alkenylene, C₁-C₂₀ alkynylene and substituted or unsubstituted C₅-C₁₀ aryl, wherein the substituents are selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], preferably L is absent;
A is an optional acidic group and each occurrence of A, if present, is independently selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], —CH₂—COOH, provided that when z and w are 0, A is present in formula IV; preferably each occurrence of A, if present, is independently selected from the group comprising H, —SO₃H, —COOH, —O—COOH, —CH₂—COOH, provided that when z and w are 0, A is present in formula IV; most preferably A is absent or occurrence of A, if present, is independently selected from the group comprising H, —COOH, —CH₂—COOH, provided that when z and w are 0, A is present in formula IV;
X⁻ is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃³¹, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate; preferably X⁻ is selected from the group comprising F⁻, HSO₄⁻, SO₄²⁻, PO₄³⁻, HPO₄²⁻, CF₃CO₂⁻, CF₃CO₃⁻, CF₃SO₃⁻; most preferably X⁻ is selected from the group comprising Cl⁻, HSO₄⁻, SO₄²⁻, CF₃SO₃⁻.
Ra is C₁-C₂₄ alkyl;
Rb and Rc are each independently selected from the group comprising H and CH₃ or absent, preferably Rc is absent;
Rd is C₁-C₂₄ alkylene or C₁-C₂₄ alkyl, optionally substituted by C₁-C₂₄ alkyl, preferably C₁-C₂ alkylene or C₁-C₂ alkyl;

Re and Rf are each independently C₁-C₂₄ alkyl, preferably CH₃;
Y is N or O, provided that when Y is O, Rc is absent;
R is selected from the group comprising C₁-C₂₄ alkyl and C₅-C₁₀ aryl or is absent.

In some embodiments the ionic polymer (IP) of the present invention, the second monomer of formula VI is

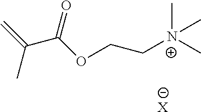

In some embodiments of the ionic polymer of the present invention, the (first) monomer of formula I is

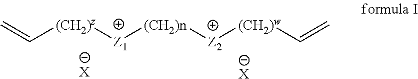

In some embodiments of the ionic polymer of the present invention, Z₁ and Z₂ are same (identical). In other embodiments, Z₁ and Z₂ are different.

In some embodiments of the ionic polymer of the present invention, when Z₁ and Z₂ is

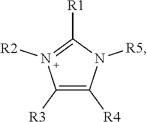

wherein R2 and R5 are bonds and R1, R3 and R4 are H, n is not 4.

In other embodiments of the ionic polymer of the present invention, when Z₁ and Z₂ is

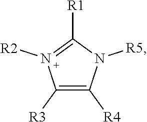

wherein R2 and R5 are bonds and n is 4, at least one of R1, R3 and R4 is not H.

In some preferred embodiments of the ionic polymer of the present invention, C₁-C₆ carboxylate are selected from the group comprising formate, acetate, propionate, butyrate, hexanoate, maleate, fumarate, oxalate, lactate, pyruvate.

The ratio between different monomers in the ionic polymers of the invention that comprises the first monomer and the second monomers can be any suitable ratio and may vary depending on the protein-containing feedstock to be processed. In some embodiments, the first and the second monomers are present in ratio 1:1.

According to some embodiments, the present invention provides monomers according to formula I selected from the group comprising

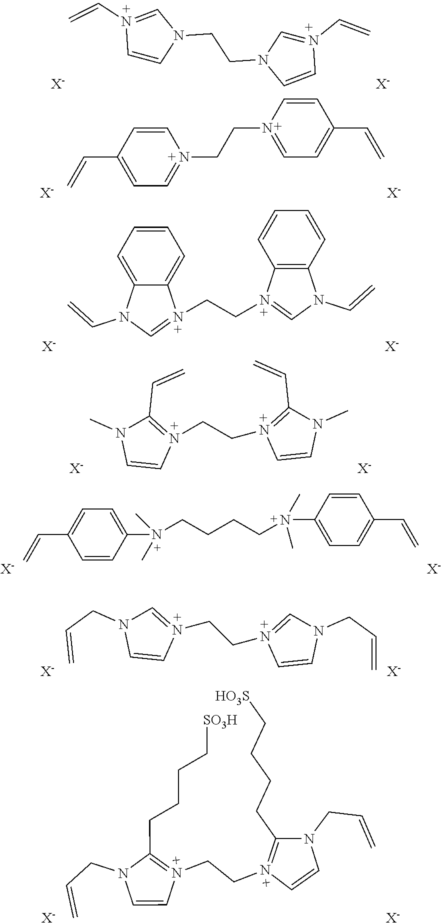

According to further embodiments, the present invention provides monomers according to formula I selected from the group comprising

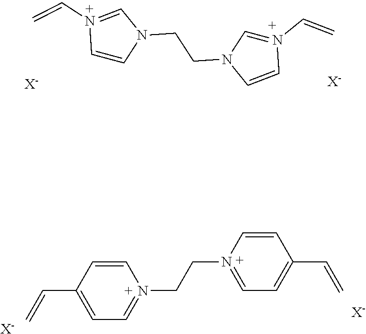

According to some embodiments, the present invention provides monomer according to formula II

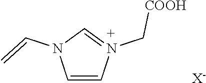

According to some embodiments, the present invention provides ionic polymers selected from the group comprising

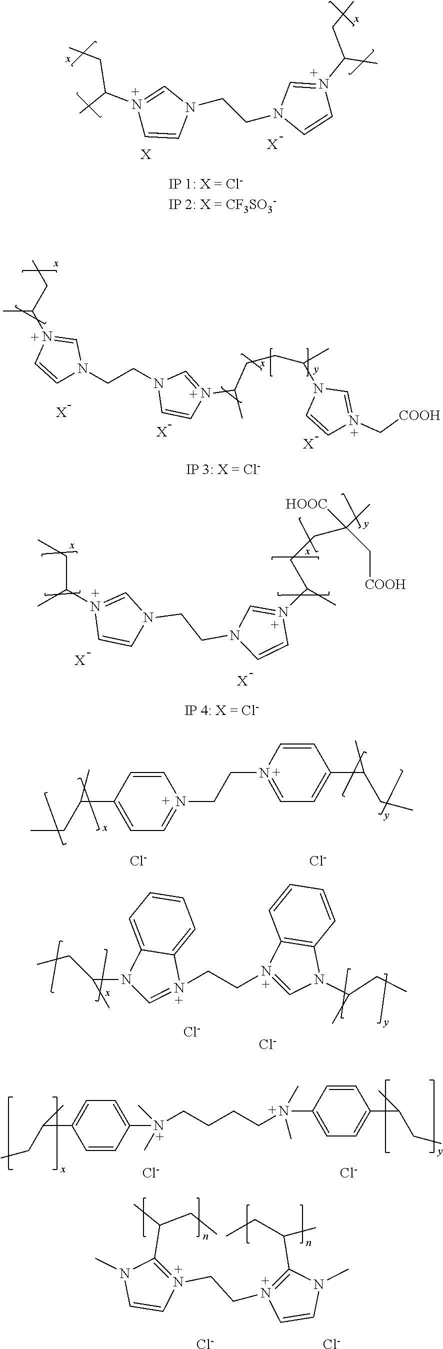

x and y are integers each independently selected within the range 1 to 1000; preferably 1 to 500 or 1 to 200; more preferably 1 to 100 or 1 to 50;

According to other embodiments, the present invention provides ionic polymers selected from the group comprising

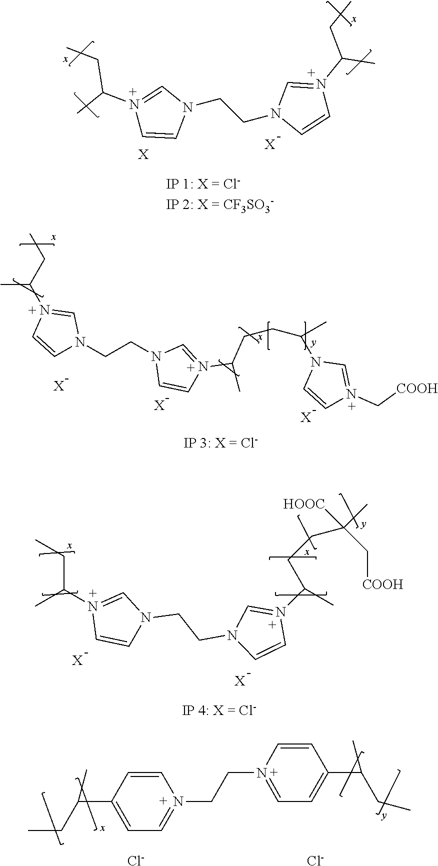

x and y are integers each independently selected within the range 1 to 1000; preferably 1 to 500 or 1 to 200; more preferably 1 to 100 or 1 to 50;

Another aspect of the present invention provides a ionic polymer (IP) consisting of a first monomer of formula I

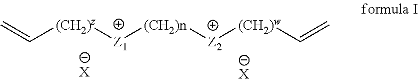

and at least one second monomer selected from the group consisting of

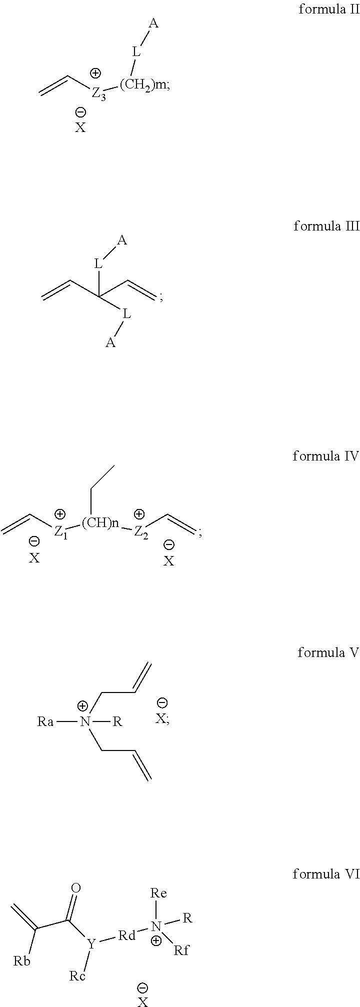

provided that at least the second monomer of formula V or at least the second monomer of formula VI is present in the ionic polymer (IP),

wherein
n and m are independently selected from 1, 2, 3, 4, 5, 6; preferably n and m are independently selected from 1, 2, 3; most preferably n is 2 and m is 1 or 2.
z and w are independently selected from 0, 1, 2, 3; preferably z and w are independently selected from 0 and 1; most preferably z and w are 0 or 1.
Z₁, Z₂ and Z₃ are cations each independently selected from the group comprising:

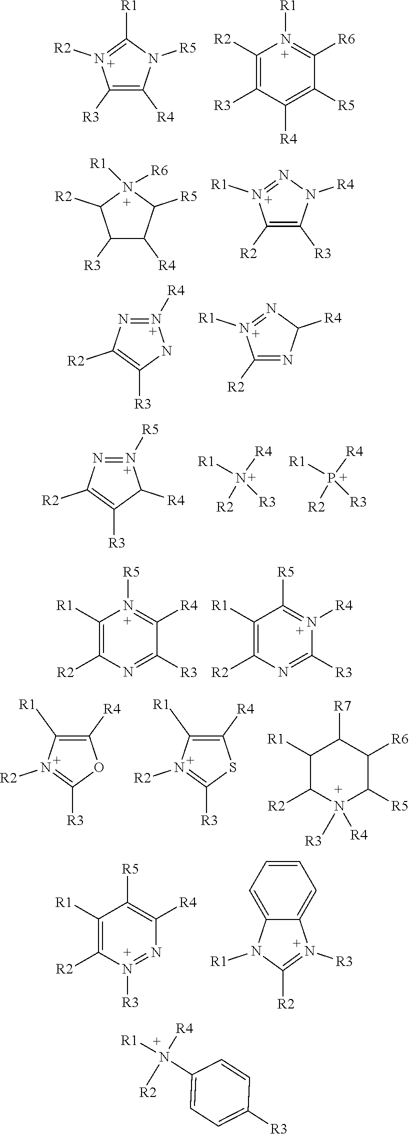

preferably Z₁, Z₂ and Z₃ are cations each independently selected from the group comprising:

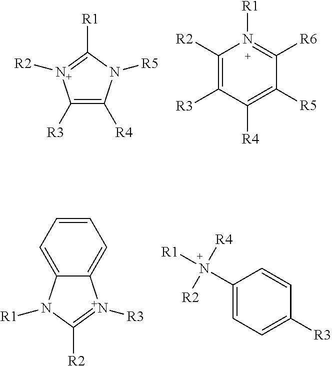

most preferably Z₁, Z₂ and Z₃ are cations each independently selected from the group comprising:

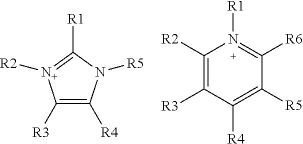

R1, R2, R3, R4, R5, R6 and R7 are each independently selected from the group comprising a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, —(CH₂)q-SO₃H, provided that two of R1, R2, R3, R4, R5, R6 and R7 are each a bond; preferably R1, R2, R3, R4, R5, R6 and R7 are each independently selected from the group comprising a bond, H, C₁-C₆ alkyl, provided that two of R1, R2, R3, R4, R5, R6 and R7 are each a bond; most preferably R1, R2, R3, R4, R5, R6 and R7 are each independently selected from the group comprising a bond and H, provided that two of R1, R2, R3, R4, R5, R6 and R7 are each a bond;
p and q are independently selected from 0, 1, 2, 3, 4, 5, 6;
L is an optional linker and each occurrence of L, if present, is independently selected from H, substituted or unsubstituted alkylene, alkenylene, alkynylene and substituted or unsubstituted C₅-C₁₀ aryl, wherein the substituents are selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], preferably L is absent;
A is an optional acidic group and each occurrence of A, if present, is independently selected from the group comprising H, —SO₃H, —COOH, —[P(═O)(OH)₂], —[P(═O)(OH)], —O—SO₃H, —O—COOH, —O—[P(═O)(OH)₂], —O—[P(═O)(OH)], —CH₂—COOH, provided that when z and w are 0, A is present in formula IV; preferably each occurrence of A, if present, is independently selected from the group comprising H, —SO₃H, —COOH, —O—COOH, —CH₂—COOH, provided that when z and w are 0, A is present in formula IV; most preferably A is absent or occurrence of A, if present, is independently selected from the group comprising H, —COOH, —CH₂—COOH, provided that when z and w are 0, A is present in formula IV;
X⁻ is selected from the group comprising F⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate; preferably X⁻ is selected from the group comprising F⁻, Cl⁻, HSO₄⁻, SO₄²⁻, PO₄³⁻, HPO₄²⁻, CF₃CO₂⁻, CF₃CO₃⁻, CF₃SO₃⁻; most preferably X⁻ is selected from the group comprising Cl⁻, HSO₄⁻, SO₄²⁻, CF₃SO₃⁻;
Ra is C₁-C₂₄ alkyl;
Rb and Rc are each independently selected from the group comprising H and CH₃ or absent, preferably Rc is absent;
Rd is C₁-C₂₄ alkylene or C₁-C₂₄ alkyl, optionally substituted by C₁-C₂₄ alkyl, preferably C₁-C₂ alkylene or C₁-C₂ alkyl;
Re and Rf are each independently C₁-C₂₄ alkyl, preferably CH₃;
Y is N or O, provided that when Y is O, Rc is absent;
R is selected from the group comprising C₁-C₂₄ alkyl and C₅-C₁₀ aryl or is absent.

In some embodiments the ionic polymer (IP) of the present invention, the second monomer of formula VI is

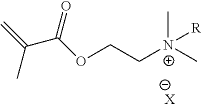

In some embodiments of the ionic polymer of the present invention, the first monomer of formula I is

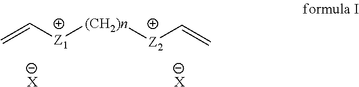

In some embodiments of the ionic polymer of the present invention, Z₁ and Z₂ are same (identical). In other embodiments, Z₁ and Z₂ are different.

In some embodiments of the ionic polymer of the present invention, when Z₁ and Z₂ is

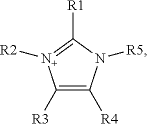

wherein R2 and R5 are bonds and R1, R3 and R4 are H, n is not 4.

In other embodiments of the ionic polymer of the present invention, when Z₁ and Z₂ is

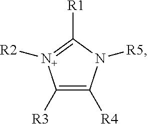

wherein R2 and R5 are bonds and n is 4, at least one of R1, R3 and R4 is not H.

Ionic polymers (IPs) of the invention can be synthesized via several methods, including but not limited to the direct polymerization of appropriate ionic species, the chemical modification of non-IPs, etc. in different solvents (water, acetonitrile, alcohols (methanol, ethanol, propanol etc.), toluene, THF) (see Examples). Polymerization may include different approaches, e.g. free radical polymerization, living/controlling radical polymerization, reversible addition-fragmentation transfer, ionic and coordination polymerization. The anionic structure can be designed according to preference before or after polymerization. The resulting ionic polymer (IP) combines the general properties of the ionic monomer and the enabling properties of a solid catalyst due to the presence of specific functional groups. In an embodiment of the invention, a salt is prepared with a cation and an anion, wherein both the cation and the anion contain vinyl groups that can be polymerized using AIBN or other initiator. It is essentially a very simple method and the ionic polymer is purified by removal of the excess AIBN by washing and filtration. In a specific embodiment of the invention, a salt that is composed of the 1-(1-vinylimidazolium)ethyl-3-vinylimidazolium] [dichloride]) is prepared. This salt, a pure compound, is then polymerized using the radical initiator AIBN. The ionic polymer is purified by removal of the excess AIBN by washing and filtration. As alternative to dichloride anion, a ditriflate anion can be obtained via anion exchange reaction prior polymerization.

The present invention also provides an ionic polymer network comprising cross-linked one or more ionic polymers of the invention.

In some embodiments, the ionic polymer network of the invention further comprises itaconic acid, citric acid and/or 1,4 butanediol.

In other embodiments, the ionic polymer network of the invention further comprises one or more metal catalysts. In some embodiments, the metal catalyst is a metal salt. In preferred embodiments anion in metal salt is selected from the group comprising F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, xylenesulfonate salts, and metal ion is selected from the group comprising Na, Ba, Sr, Ca, Cd, Sn, Pb, Fe, Cu, Zn, Zr, Mn, Co, Ni, Li, Al, Cr, Mg, Mo, Hg, Ag, Au, Pt, Rh, Re, Ti, Pb, Bi, Ga, In, Sn, Ir, La, Hf, Ta, W, Os.

In some preferred embodiments, C₁-C₆ carboxylate are selected from the group comprising formate, acetate, propionate, butyrate, hexanoate, maleate, fumarate, oxalate, lactate, pyruvate.

The ionic polymer network of the invention comprising one or more metal catalysts provides better stability and reusability of the ionic polymer-metal combinations.

The preparation of the ionic polymer network of the invention with one or more metal catalysts typically consists in mixing or refluxing the ionic polymer network and metal salt in water/organic solvent overnight. See for example J. Am. Chem. Soc., 2012, 134, 11852-11855; Chem. Cat. Chem., 2016, 8, 2508-2515; J. Org. Chem., 2011, 76 (24), pp 10140-10147; Inorg. Chem., 2006, 45, 6396-6403.

The ionic polymers of the invention can be incorporated in membranes or attached to solid supports.

Another aspect of the invention provides membranes composed of ionic polymers of the invention. In some embodiments, the invention provides a polymer membrane comprising one or more ionic polymers of the invention. By adding appropriate copolymer (for example acrylic acid) to the salt used for preparation of ionic polymers of the invention and then polymerize the mixture it is possible to generate a polymer membrane. An approach for membrane formation is based on the template-free method via simple ionic complexations when an ionic monomer is copolymerized with appropriate organic acid/acid derivative (see Tauber K. et al, Polym. Chem., 2015, 6, 4855-4858; Tauber K. et al, ACS Macro Lett., 2015, 4(1), 39-42; Zhang S. et al, Chem. Sci., 2015, 6, 3684-3691). As example, ionic monomer was dissolved in DMSO and stirred for 2 h at 60° C. The transparent solution was then poured onto a glass plate and the solvent was evaporated at 80° C. in an oven. The resulting non-porous dry polymer film was subsequently immersed into aqueous ammonia (0.2 wt %) overnight for pore formation and electrostatic complexation. The membrane was detached easily from the glass plate and washed several times with water.

Another aspect of the invention provides solid-supported ionic polymers. In some embodiments, the invention provides a solid support having at least one surface comprising one or more ionic polymers of the invention. Supported ionic polymers can be immobilized on different materials as a support: silicon or carbon (nanotube, wire) source, graphene or graphene oxide, zeolites, metal/metals alloys or metal/metal alloy oxides. As example, FeOₓ support has been oxidized in the oven in presence of oxygen at high temperature (500° C.) and its surface was modified with mixture of silanes dissolved in ethanol in presence of HCl afterwards. After drying at room temperature the support was uniformly impregnated with methanol solution of ionic polymer and AIBN. After drying at room temperature, the obtained material was placed in the oven at 95° C. for 2 h. By repeating the impregnation process the desire polymer loading might be achieved. Another example is stainless steel membrane comprising ionic polymers of the invention. A mixture containing ionic monomer (0.2-0.5, molar ratio), acrylic acid (0.1-0.6, molar ratio), and benzoin ethylether (1 wt %, as a photo-initiator) were dissolved in methanol to achieve a homogeneous solution, which was then dispersed by wettening onto stainless steel membrane and photo-crosslinked at room temperature by irradiation with UV light of 250 nm wavelength.

Ionic polymer attachment is also possible through surface grafting, which requires activation of the support by UV or O₃, O₂, H₂ or air plasmas. It involves the creation of reactive sites (radicals) on the polymer surface followed by the covalent linkage of a preformed polymer or, more commonly, by the polymerization of a monomer from those radical sites (see Alves P. et al, Colloids and Surfaces B: Biointerfaces, Volume 82, Issue 2, 1 Feb. 2011, 371-377; Barbey R. et al., Chem. Rev., 2009, 109(11), 5437-5527). Another copolymer or polymerization initiator might also be used during the polymerisation process (as in case of membrane formation).

In some embodiments of the method for hydrolysing proteins and protein-containing feedstock into protein hydrolysate of the present invention, the organic solvent is selected from the group comprising alcohol (such as methanol, ethanol, butanol, ethylene glycol, etc.), ether (such as dimethoxyethane, diglyme, butyl methyl ether, etc.), ketone (such as methyl isobutyl ketone, N-methyl-2-pyrrolidone, etc.), eutectic solvents (such as glycerol—choline chloride, octanoic acid—tetrabutylammonium chloride, poly (ethylene glycol)—choline chloride, lactic acid—glycine).

In some embodiments of the methods of the present invention, recovering the protein hydrolysate can be done by any technic known in the art, such as filtration, centrifugation or gravity settling.

The effective amount of the ionic polymers of the invention or a combination thereof used in the methods described herein can depend on several factors including, for example, the type of the protein or the protein-containing feedstock, the amount of the protein-containing feedstock, the content of proteins in the protein-containing feedstock, the type and number of pre-treatment(s) applied to the protein-containing feedstock, and the reaction conditions (such as temperature and time). An effective amount of the ionic polymer of the invention refers to an amount sufficient to degrade the proteins or the protein-containing feedstock into the protein hydrolysate of the invention. In some embodiments, the effective amount of the ionic polymer of the invention is usually 0.05:1 w/w to 10:1 w/w, 0.5:1 w/w to 10:1 w/w, 1:1 w/w to 1:5 w/w, preferably 0.1:1 w/w to 1:5 w/w compared to protein content in the protein-containing feedstock.

The ratio protein-containing feedstock to water used in the methods described herein can depend on several factors, including for example the type of the protein-containing feedstock and the amount of the protein-containing feedstock. In some embodiments, the ratio the protein-containing feedstock to water or organic solvent (such as alcohol, ether, ketone, eutectic solvent) used in the methods described herein is ranging from 1:100 w/v to 1:1 w/v, preferably 1:50 w/v to 1:10 w/v.

The preferred temperature profile for the heating used in the methods described herein depends on the protein-containing feedstock starting material being used and also the intended protein hydrolysate being produced. The heating temperature should preferably be held at a maximum of 200° C., in some embodiments at a maximum of 170° C. or 160° C. In some embodiments, the heating temperature is between 100° C. and 200° C., or between 100° C. and 170°, or between 100° C. and 160° C., or between 110° C. and 150° C.; preferably between 120° C. to 170° C. or between 120° C. to 140° C. Preferably, for small-scale applications, the heating is done in a high-pressure autoclave reactor, which after sealing, is heated for appropriate reaction time and temperature.

The method for producing the protein hydrolysate of the present invention operates at moderate temperatures, typically less than 170° C. or 160° C. or even less than 50° C., whereas the prior art methods needs temperatures of more than 170° C. In addition, the method for producing the protein hydrolysate of the present invention provides fewer by-products, which allows easier recovery of the desired products.

In some embodiments, the appropriate reaction time in the methods described herein is for example between 10 minutes and 10 hours, preferably between 0.5 hour and 5 hours or between 1 hour and 3 hours, depending on the type and amount of the protein-containing feedstock.

An important advantage of the ionic polymers of the invention or a combination thereof, membranes incorporating ionic polymers of the invention and/or solid-supported ionic polymers of the invention and use thereof for protein and protein-containing feedstock hydrolysis, decomposition or degradation is their use in one-pot systems for decomposition and selective extracting the aforementioned protein hydrolysate from the protein-containing feedstock. Further, ionic polymers of the invention are insoluble, thus does not mix with the protein hydrolysate of the present invention.

According to the present invention, to make proteins a more appealing food substitute having improved digestibility, absorption and benefits, the large native proteins are hydrolysed into smaller protein fragments called protein hydrolysates, such as peptides and/or amino acids, with ionic polymers disclosed herein.

Another aspect of the present invention provides a protein hydrolysate obtained by the method for hydrolysing proteins and/or protein-containing feedstock of the present invention. In some embodiments, the protein hydrolysate obtained by the method of the invention comprises peptides and free amino acids, wherein the protein hydrolysate is water soluble, having improved dissolving properties and degree of hydrolysis above 2.5% and wherein peptides are oligopeptides and polypeptides, optionally linked to carbohydrates, having a molecular weight of less than 10′000 Da.

Another aspect of the present invention provides a protein hydrolysate comprising peptides, such as oligopeptides and polypeptides, and free amino acids. In some embodiments, the peptides present in the protein hydrolysate of the present invention are glycopeptides and/or peptides linked to carbohydrates.

In some embodiments, the oligopeptides and/or the polypeptides in the protein hydrolysate have a molecular weight of less than 10′000 Da, preferably of 500 to 8000 Da.

The peptides typically contain 2 to 20 amino acids or more. The peptides can be bioactive. The protein hydrolysate of the invention is water soluble, having improved dissolving properties and degree of hydrolysis above 2.5%

In some embodiments, the protein hydrolysate of the present invention comprises peptides and free amino acids, wherein the protein hydrolysate is water soluble, having improved dissolving properties and degree of hydrolysis above 2.5% and wherein peptides are oligopeptides and polypeptides, optionally linked to carbohydrates, having a molecular weight of less than 10′000 Da.

In other embodiments, the protein hydrolysate of the present invention consists of peptides and free amino acids, wherein the protein hydrolysate is water soluble, having improved dissolving properties and degree of hydrolysis above 2.5% and wherein peptides are oligopeptides and polypeptides, optionally linked to carbohydrates, having a molecular weight of less than 10′000 Da.

In some embodiments, the protein hydrolysate is obtained by the method for hydrolysing proteins and/or protein-containing feedstock of the present invention.

The protein hydrolysate of the present invention can act as physiological modulators of metabolism and can possess a wide range of bioactivities including immunomodulatory, anticancer, antihypertensive, antioxidant, anti-inflammatory, mineral binding, opiate, antilipemic, antimicrobial/antibacterial, antifungal and antiviral agents, anticoagulants, thermogenic agents, anti-osteoporosis (osteoprotective), cell growth and repair modulators, angiotensin-converting enzyme (ACE) inhibitors, in addition to biological signalling mediators involved in a myriad of signalling functions with impact on recovery, lipid metabolism, carbohydrate metabolism, immune function, cardiovascular and bone health, nervous system and brain function, optimizing muscle performance during exercise, digestive satiety and weight management.

In addition to bioactivities, the protein hydrolysate of the present invention possess various physicochemical properties including solubility, lipid binding, foaming, and emulsification properties depending on their composition, sequence, and length. Indeed, the protein hydrolysate of the present invention can exert beneficial effects on improving intestinal morphology, function, and resistance to infectious diseases in humans and animals (such as pigs, calves, chickens, companion animals and fish), thereby enhancing their health and well-being, as well as growth performance and feed efficiency. This provides a cost-effective approach to converting protein-containing feedstock, such as animal by-products, brewer's by-products, or plant feedstuffs, into high-quality protein-hydrolysate ingredients to provide human dietary supplements and animal feed formulations. Further, the protein hydrolysate of the present invention can have applications in textiles including applications such as plywood adhesives; aquaculture and agriculture including applications such as promotion of plant rooting, germination, growth and prolong lifetime; cosmetic formulation; biopharmaceuticals and absorbent hydrogel formulation; functional food and beverage formulation; enteric diet formulation and dietary supplementation; infant and paediatric nutritional product formulation; animal feed formulation; cell culture growth medium and fermentation processing; baking ingredient to improve resistance against freezing and favourable texture.

The protein hydrolysate of the present invention can be used in various applications selected from the group comprising pharmaceutical, preventative health, dietary supplement, functional food and beverage, paediatric nutrition, food additive, animal feed, fertilizer, antioxidant, antimicrobial, cosmetic, surfactant applications.

Preferably, the protein hydrolysate of the present invention is used in the manufacturing of a food product, a functional food, a nutritional supplement, an animal feed product and/or cosmetic product.

In another embodiment, the present invention provides a method for manufacturing a food product, a functional food, a nutritional supplement, an animal feed product and/or cosmetic product, wherein the method comprises the use of the protein hydrolysate of the present invention.

Another aspect of the present invention provides a food product, a functional food, a nutritional supplement, an animal feed product and/or cosmetic product comprising the protein hydrolysate of the present invention.

In some embodiments of the present invention, the food product comprises an edible material and the protein hydrolysate of the present invention. The selection of a particular protein hydrolysate to combine with an edible material can and will vary depending upon the desired food product. The selection of the appropriate edible material also will vary depending on the desired food product. The edible material may be a plant-derived material (for example a vegetable juice, a cereal product, etc.), an animal-derived material (for example a dairy product, an egg product, etc.), or a biomaterial (for example a protein, a carbohydrate, a lipid, etc.) isolated from a plant-derived material or an animal-derived material, and so forth.

The food product of the present invention may include, for example, hot or cold cereals, bars, baked goods, beverages, yogurts, desserts, snacks, pastas, and meats (including poultry and seafood).

The protein hydrolysate of the present invention can be combined with all kinds of ingredients such as oils, fats, emulsifiers, carbohydrates, fruit concentrates, flavours, colorants, alcohol, carbon dioxide, thickeners, acidulates, antioxidants, herbs or herb extracts, health promoting compounds like vitamins or bioactive compounds to formulate a product which is in line with the marketing needs.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### EXAMPLES

Preparation of Monomer of Formula VI

To prepare cationic DMAEMAQ 30 mmol of DMAEMA were dissolved in an appropriate amount of solvent (solvent may be THF, acetonitrile, DCM, chloroform, diethyl ether, etc.) followed by careful addition of 36 mmol of RX, where X may be halide, HSO4- or SO42- and R is alkyl or aryl. The reaction mixture was stirred at room temperature for 12 h. Afterwards, the precipitate was filtered and washed with addition of cold hexane. Finally, the obtained product was dried for 8 h under vacuum to yield the crystalline solid of corresponding DMAEMAQ monomer.

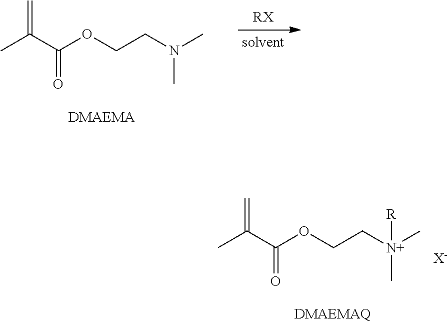

Preparation of Protein Hydrolysate from Whey Gluten

5 g of whey gluten suspended in 75 ml of water together with 65 mg of IP1 were stirred at 140° C. for 1 h. After reaction, the mixture was cooled to room temperature, filtered, solid phase was dried and the liquid phase was subjected for QTOF-ESI(+) analysis. 35% weight conversion was measured.

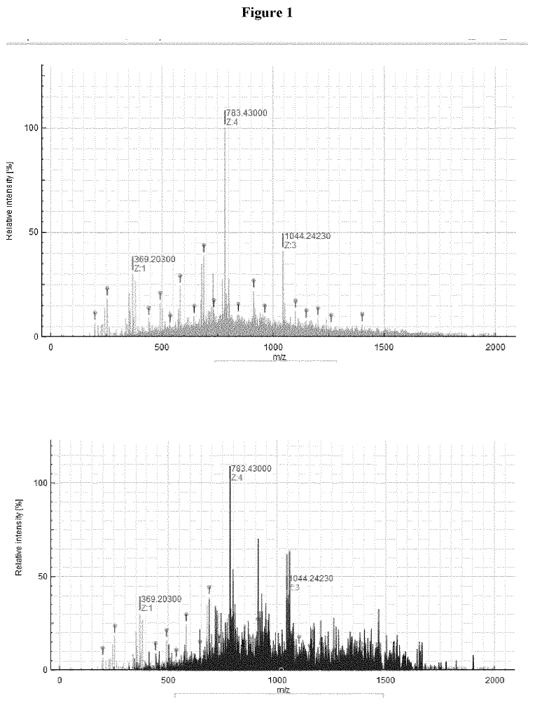

The interpretation of the obtained data reviled the mixture of oligopeptides and polypeptides with the mass range 500-7000 Dalton. See FIG. 1.

Preparation of Protein Hydrolysate from Bovine Albumin

100 mg of bovine albumin suspended in 1.5 ml of water together with 4 mg of IP1 were stirred at 140° C. for 1 h. After reaction, the mixture was cooled to room temperature, filtered and the liquid phase was subjected for QTOF-ESI(+) analysis.

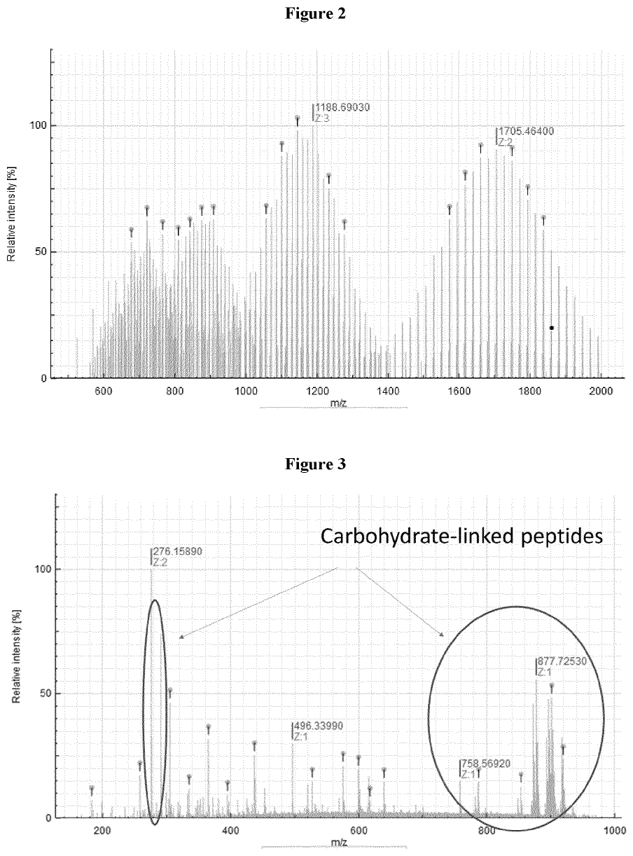

The interpretation of the obtained data reviled the mixture of oligopeptides and polypeptides with the mass range 500-8000 Da. See FIG. 2.

Preparation of Protein Hydrolysate from Spent Barley

200 g of spent barley suspended in 1,5 L of water together with 3 g of IP1 were stirred at 140° C. for 1 h. After reaction, the mixture was cooled to room temperature, filtered, and the liquid phase was subjected for QTOF-ESI(+) analysis.

The interpretation of the obtained data reviled the presence of peptides linked to carbohydrates. See FIG. 3.

Preparation of Protein Hydrolysate from Yeast Slurry

67 g of yeast slurry (dry bases) suspended in 0,666 L of water together with 0,22 g of IP3 were stirred at 145° C. for 1,5 h. After reaction, the mixture was cooled to room temperature, filtered and the liquid phase was dried using spray drier. The obtained dried product was analysed for protein content using Kjeldahl's method and 45 wt % as hydrolysed protein was measured.

### Example of Physicochemical Improvement of the Proteins

Okara has a high nutritive value due to its high-quality protein (15.2-33.4%), fat (8.3-10.9%), carbohydrates and fibre (30-58%). Due to the technological process, the okara residue contains predominantly insoluble or non-extractable proteins. The okara protein isolates contain all of the essential amino acids and have a protein efficiency index that is even higher than that of soymilk (2.71 vs. 2.11) and tofu, but with low water solubility. It has also been found that the protein fraction of okara is able to withstand complete digestion by the gastrointestinal enzymes, pepsin and pancreatin (the latter of which mainly consists of trypsin, amylopsin and steapsin). However, the unwanted flavour of okara, called the “beany” flavour in their research, was a major challenge for its application and production of okara-based food products. To address such challenge, a hydrolysis treatment may be applied resulting in an increase of the soluble fibre and soluble protein content, and thus improve nutritional quality and processing properties.

Preparation of Okara Hydrolysate I:

10 g of okara (dry bases) suspended in 100 ml of water together with ionic polymer catalyst IP3 were stirred at 120° C. for 1 h. After reaction, the mixture was cooled to room temperature, filtered and the liquid phase was dried using spray drier.

Preparation of Okara Hydrolysate II:

10 g of okara (dry bases) suspended in 100 ml of water together with ionic polymer catalyst IP3 were stirred at 130° C. for 1 h. After reaction, the mixture was cooled to room temperature, filtered and the liquid phase was dried using spray drier.

| Main analysed | Okara | Okara | Okara |
|---|---|---|---|
| components, g/100 g | feedstock | hydrolysate I | hydrolysate II |
| glucose | 2.3 | 3.6 | 2.2 |
| xylose | 7.2 | 2.9 | 2.9 |
| galactose | 13.2 | 14.8 | 18.6 |
| arabinose | 5.8 | 9.3 | 9.6 |
| mannose | 5.7 | 6.8 | 4.5 |
| total | 34.2 | 37.4 | 37.8 |
| protein hydrolysate | 29 | 19.5 | 21 |

### Example of the Beneficial Effect on Specific Bacteria Growth

Macronutrients and micronutrients can modulate the microbiota. The overall balance between the macronutrients such as protein, carbohydrate and fat is known to influence the composition and functional potential of the gut microbiota. The efficacy of spent barley hydrolysate (the composition Prembion comprising the protein hydrolysate of the invention) was compared to Glucose, a commercial XOS and FOS products and was tested measuring the growth curves of a Bifidobacterium X and Lactobacillus X bacterial strains, which activity has been linked to improvements in animal and human gut health.

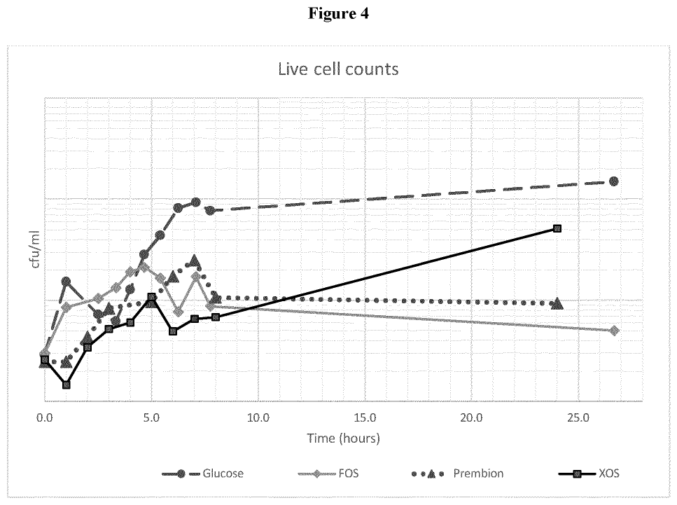

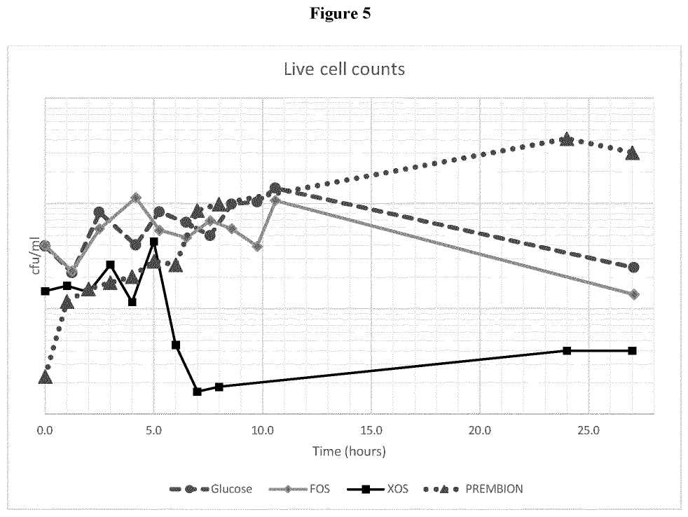

FIGS. 4 and 5 shows how two different strains perform in vitro when media is supplemented with different nutrient sources. When compared to pure FOS and XOS, a complex product derived from spent barley and containing hydrolysed protein demonstrated superior preferential growth of Bifidobacterium strain (FIG. 5) and comparative growth of Lactobacillus strain (FIG. 4).

1. A method for hydrolyzing proteins and/or protein-containing feedstock into a protein hydrolysate, the method comprising the steps of:
a) providing protein and/or protein-containing feedstock;
b) optionally determining protein content in the protein-containing feedstock;
c) optionally pre-treating the protein-containing feedstock;
d) optionally isolating proteins from the protein-containing feedstock to form a protein concentrate;
e) contacting the protein, the protein concentrate and/or the protein-containing feedstock with a catalyst to form a reaction mixture, wherein the catalyst is an ionic polymer or a combination of ionic polymers, an ionic polymer network, a solid-supported ionic polymers and/or a polymer membrane incorporating ionic polymers;
f) degrading the protein, the protein concentrate and/or the protein-containing feedstock in the reaction mixture to produce a liquid phase and a solid phase, wherein the liquid phase includes the protein hydrolysate, and the solid phase includes residual materials;
g) isolating at least a portion of the liquid phase from the solid phase; and
h) recovering the protein hydrolysate from the isolated liquid phase;
wherein the ionic polymer (IP) consists of a monomer of formula I 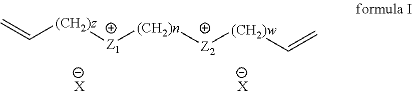
or consists of a first monomer of formula I 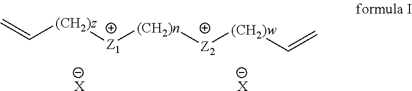
and at least one second monomer selected from the group consisting of 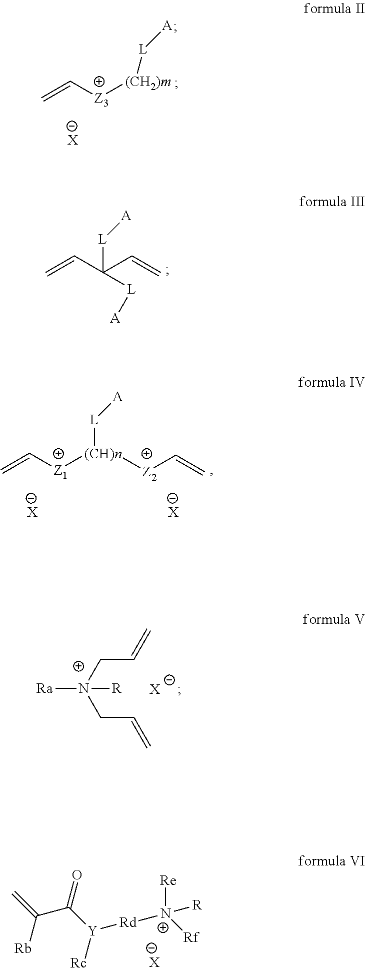
wherein
n and m are independently selected from 1, 2, 3, 4, 5, and 6;
z and w are independently selected from 0, 1, 2, and 3;
Z₁, Z₂ and Z₃ are cations each independently selected from the group consisting of: 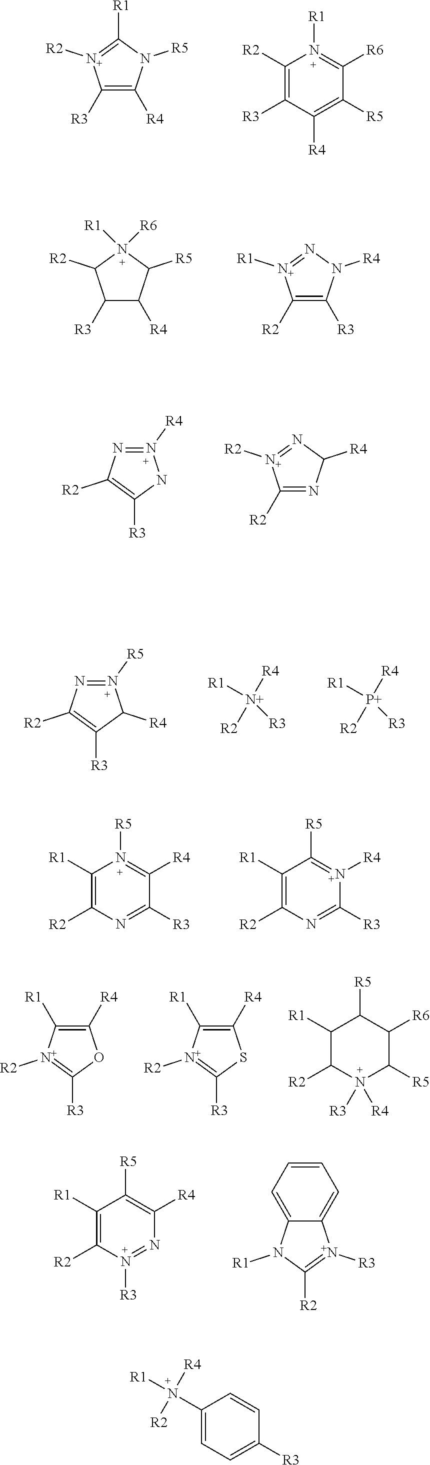
wherein R1, R2, R3, R4, R5, R6 and R7 are each independently selected from the group consisting of a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, and —(CH₂)q-SO₃H, provided that two of R1, R2, R3, R4, R5, R6 and R7 are each a bond;
p and q are independently selected from 0, 1, 2, 3, 4, 5, and 6;
L is an optional linker and each occurrence of L, if present, is independently selected from H, substituted or unsubstituted C₁-C₂₀ alkylene, C₁-C₂₀ alkenylene, C₁-C₂₀ alkynylene, and substituted or unsubstituted C₅-C₁₀ aryl, wherein the substituents are selected from the group consisting of H, —SO₃H, —COOH, —[P(═O)(OH)₂], —[P(═O)(OH)], —O—SO₃H, —O—COOH, —O— [P(═O)(OH)₂], and —O—[P(═O)(OH)];
A is an optional acidic group and each occurrence of A, if present, is independently selected from the group consisting of H, —SO₃H, —COOH, —[P(═O)(OH)₂], —[P(═O)(OH)], —O—SO₃H, —O—COOH, —O—[P(═O)(OH)₂], —O—[P(═O)(OH)], and —CH₂—COOH, provided that when z and w are 0, A is present in formula IV;
X⁻ is selected from the group consisting of F⁻, Cl⁻, Br, I⁻, ClO₄⁻, BF⁻, PF₆⁻, AsF₆⁻, SbF₆⁻, NO₂⁻, NO₃⁻, HSO₄⁻, SO₄²⁻, PO₄³⁻, HP₃O₄²⁻, CF₃CO₂⁻, CF₃CO₃⁻, CO²⁻, CF₃SO₃⁻, C₁-C₆carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, and xylenesulfonate;
Ra is C₁-C₂₄ alkyl;
Rb and Rc are each independently selected from the group consisting of H and CH₃ or Rb and Rc are each independently absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is selected from the group consisting of C₁-C₂₄ alkyl and C₅-C₁₀ aryl, or R is absent,
wherein the ionic polymer network comprises cross-linked the one or more ionic polymers (IP);
wherein the solid support has at least one surface comprising the one or more ionic polymers (IP) or the ionic polymer network;
wherein the polymer membrane incorporates the one or more ionic polymers (IP) or the ionic polymer network.

2. The method of claim 1, wherein in the ionic polymer, Z₁, Z₂ and Z₃ are cations and each independently selected from the group consisting of:

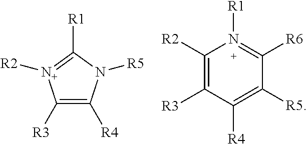

3. The method of claim 1, wherein the second monomer of formula VI is

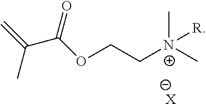

4. A protein hydrolysate obtained by the method of claim 1.

5. The protein hydrolysate of claim 4, comprising peptides and free amino acids, wherein the protein hydrolysate is water soluble, having improved dissolving properties and degree of hydrolysis above 2.5%, and wherein the peptides are oligopeptides and polypeptides, optionally linked to carbohydrates, having a molecular weight of less than 10′000 Da.

6. (canceled)

7. A food product comprising an edible material and the protein hydrolysate of claim 4.

8. An ionic polymer (IP) consisting of a first monomer of formula I 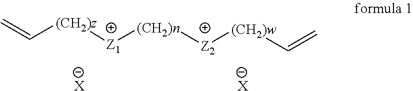
and at least one second monomer selected from the group consisting of 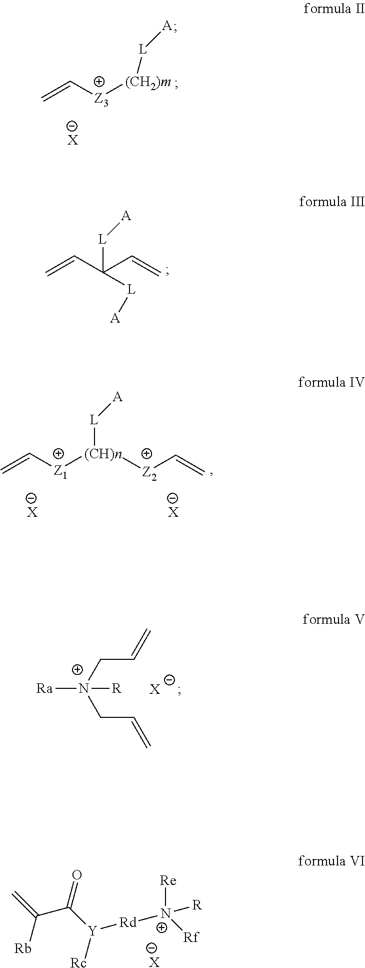
provided that at least the second monomer of formula V or at least the second monomer of formula VI is present in the ionic polymer (IP), wherein
n and m are independently selected from 1, 2, 3, 4, 5, and 6;
z and w are independently selected from 0, 1, 2, and 3;
Z₁, Z₂ and Z₃ are cations each independently selected from the group comprising: 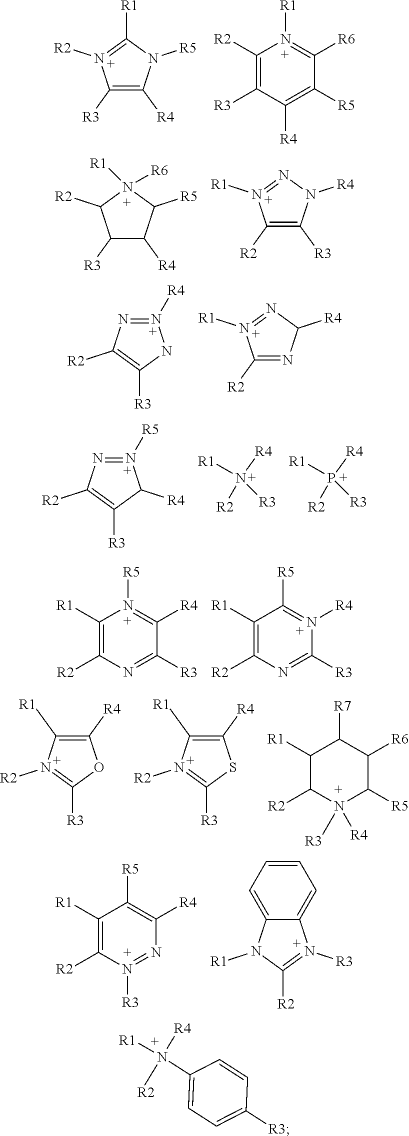
wherein R1, R2, R3, R4, R5, R6 and R7 are each independently selected from the group consisting of a bond, H, C₁-C₆ alkyl, C₁-C₆ allyl, CH₃—(CH₂)p-O—(CH₂)q-CH₃, C₁-C₆ alkoxy, C₁-C₆ alkoxyalkyl, benzyl, —SO₃H, and —(CH₂)q-SO₃H, provided that two of R1, R2, R3, R4, R5, R6 and R7 are each a bond;
p and q are independently selected from 0, 1, 2, 3, 4, 5, and 6;
L is an optional linker and each occurrence of L, if present, is independently selected from H, substituted or unsubstituted C₁-C₂₀ alkylene, C₁-C₂₀ alkenylene, C₁-C₂₀ alkynylene, and substituted or unsubstituted C₅-C₁₀ aryl, wherein the substituents are selected from the group consisting of H, —SO₃H, —COOH, —[P(═O)(OH)₂], —[P(═O)(OH)], —O—SO₃H, —O—COOH, —O— [P(═O)(OH)₂], and —O—[P(═O)(OH)];
A is an optional acidic group and each occurrence of A, if present, is independently selected from the group consisting of H, —SO₃H, —COOH, —[P(═O)(OH)₂], —[P(═O)(OH)], —O—SO₃H, —O—COOH, —O—[P(═O)(OH)₂], —O—[P(═O)(OH)], and —CH₂—COOH, provided that when z and w are 0, A is present in formula IV;
X⁻ is selected from the group consisting of F⁻, Cl⁻, Br⁻, I⁻, ClO₄⁻, BF₄⁻, PF₆⁻, AsF₆⁻, SbF₆⁻, NO₂⁻, NO₃⁻, HSO₄⁻, SO₄²⁻, PO₄³⁻, HPO₄²⁻, CF₃CO₂⁻, CF₃CO₃⁻, CO₃²⁻, CF₃SO₃⁻, C₁-C₆ carboxylate, CN⁻, SCN⁻, OCN⁻, CNO⁻, N₃⁻, tosylate, mesylate, trifluoromethanesulfonate, trifluoroethane sulfonate, di-trifluoromethanesulfonyl amino, docusate, and xylenesulfonate;
Ra is C₁-C₂₄ alkyl;
Rb and Rc are each independently selected from the group comprising H and CH₃, or Rb and Rc are each independently absent;
Rd is C₁-C₂₄ alkylene or C₁-C₂₄ alkyl, optionally substituted by C₁-C₂₄ alkyl;
Re and Rf are each independently C₁-C₂₄ alkyl;
Y is N or O, provided that when Y is O, Rc is absent;
R is selected from the group comprising C₁-C₂₄ alkyl and C₅-C₁₀ aryl, or R is absent.

9. The ionic polymer of claim 8, wherein Z₁, Z₂ and Z₃ are cations and each independently selected from the group consisting of:

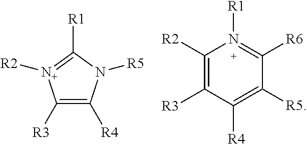

10. The ionic polymer of claim 8, wherein the second monomer of formula VI is [chemical structure]
